(19) United States
(12) Patent Application Publication
(10) Pub. No.: US 2015/0150487 A1
(43) Pub. Date: Jun. 4, 2015

(54) Method and system for optimized selection of a bicycle saddle pertaining to a population of different saddles

(71) Applicant: ERGOVIEW AG, Chur (CH)

(72) Inventor: Giuseppe Bigolin, Rossano Veneto (VI) (IT)

(73) Assignee: ERGOVIEW SRL, Cesena (IT)

(21) Appl. No.: 14/406,700

(22) PCT Filed: Jun. 11, 2013
(86) PCT No.: PCT/IB2013/054775

§ 371 Date: Dec. 9, 2014

(30) Foreign Application Priority Data
Jun. 11, 2012 (CH) 813/12
Jun. 10, 2013 (CH) 1087/13

(51) Int. Cl. A61B 5/107; G01C 9/02

(52) U.S. Cl. CPC A61B 5/107; G01C 9/02

(57) ABSTRACT

A method for optimized selection of a bicycle saddle from a population of different bicycle saddles includes the steps of providing a database composed of a set of physical elements corresponding to the population of different saddles, subdividing the set into a plurality of subsets according to predetermined geometrical and/or functional characteristics, collecting a plurality of anatomical parameters of a user to obtain corresponding data, and selecting one of the subsets having corresponding optimized characteristics according to the data. The geometrical and/or functional characteristics are selected from the group that includes the maximum plan width, the presence and dimension of holes and/or superficial hollows, and the plan shape of the saddles. The parameters are collected on the user in an upright position and with the legs stretched. A system for anthropometric determination of one or more optimized bicycle saddles.

### FIELD OF THE INVENTION

The present invention generally finds application in the field of human body support devices, and particularly relates to a method of optimized selection of a bicycle saddle according to anthropometric data.

The invention also relates to a system for carrying out the above method using suitable devices and equipment.

### BACKGROUND ART

A multiplicity of saddles are known to be commercially available, which differ in terms geometrical, structural and/or functional characteristics.

Therefore, it is important for a user to be able to select the most appropriate saddle based not only on preferred seating conditions, but also on his/her own anatomical and anthropometric characteristics, such that the saddle can ensure optimal comfort and achievement of the required performance.

A method of selecting an optimized saddle according to the anatomic characteristics of a particular user is known, for instance, from U.S. Pat. No. 7,284,336.

In this known method, the sit-bone distance of each particular user, i.e. the distance between his/her ischial tuberosities, is determined for proper selection of a saddle having a rear portion of appropriate width, which ensures proper support also to heavy-set users.

Particularly, the sit-bone distance is measured by making the saddle user sit on a gel-padded cushion, such that his/her ischial tuberosities leave temporary impressions for such measurement.

Saddle width selection also depends on the preferred posture of the user as he/she rides, such that users having the same sit-bone distance but different postures, e.g. with a substantially straight or forward-leaning torso, may be associated with saddles of different widths.

The above prior art method did not prove to be fully satisfactory, as it considers limited anatomic characteristics and does not allow selection of saddles having additional anthropometrically optimized characteristics, other than the maximum width.

Furthermore, the selection of a maximum saddle width also according to the user-preferred riding posture has a poor scientific value, and is a merely empirical aspect, that does not change the comfort conditions provided by saddles with different widths.

### DISCLOSURE OF THE INVENTION

A general object of the present invention is to solve the above technical problem, by providing a method for optimized selection of a bicycle saddle that is particularly suitable and effective in both anthropometric and functional terms.

A particular object is to provide a method for optimized selection of a bicycle saddle that allows a saddle or a group of saddles to be singled out from a relatively wide population of saddles, as having particular geometrical and/or functional features optimized according to a plurality of anatomical parameters of the user.

Another object of the present invention is to provide a method for optimized selection of one or more bicycle saddles that allows each user to be associated with the best available saddle, irrespective of the posture assumed by the user as he/she rides the bicycle.

A further object of the present invention is to provide a method for optimized selection of a bicycle saddle that ensures the best comfort and the best conditions to achieve high performance, for each particular user.

Yet another object of the invention is to provide a system for optimized anthropometric selection of a bicycle saddle that ensures the utmost accuracy and reliability.

Another object of the invention is to provide a system for optimized anthropometric selection of a bicycle saddle that is quick and easy to use, preferably by the user him/herself, without requiring the assistance of any external personnel.

These and other objects, as better explained hereafter, are fulfilled by a method for optimized anthropometric selection of a bicycle saddle according to the main claim.

Thanks to this sequence of measurements, and to the combination of data with geometrical and/or functional characteristics of the saddle, it is possible to obtain optimal and accurate determination of the saddle having all the technical characteristics that ensure comfort, irrespective of the riding posture assumed by the user.

In a further aspect, the invention relates to a system for optimized anthropometric selection of a bicycle saddle as defined in the independent claim 17.

### BRIEF DESCRIPTION OF THE DRAWINGS

Further characteristics and advantages of the invention will be more apparent from the detailed description of a few preferred, non-exclusive embodiments of a method and a system for optimized selection of a bicycle saddle according to the invention, which are described as non-limiting examples with the help of the annexed drawings, in which:

FIG. 1 is a flow chart of a general implementation of the selection method of the invention;

FIG. 2 is a perspective view of a first embodiment of a selection system for carrying out the method of the invention;

FIG. 3 is a top view of the system of FIG. 2;

FIG. 4 is a front view of the system of FIG. 2;

FIG. 5 is a side view of the system of FIG. 2;

FIGS. 6 to 9 show various steps of the method that uses the system as shown in FIGS. 2 to 5;

FIG. 10 shows a flow chart of a particular implementation of the selection method of the invention;

FIG. 11 is a three-dimensional view of the set of physical elements corresponding to saddles that belong to a given initial population;

FIG. 12 is a plan view of a few saddles that belong to a set of physical elements that can be used for carrying out the method;

FIG. 13 is a flow diagram of the selection method as schematically shown in FIG. 10;

FIG. 14 is a general view of the system of the invention;

FIG. 15 is a schematic perspective view of a first step for measuring a first characteristic of the method of FIG. 10;

FIG. 16 is a schematic perspective view of a second step for measuring a second characteristic of the method of FIG. 10;

FIG. 17 is a schematic perspective view of a third step for measuring a third characteristic of the method of FIG. 10;

FIG. 18 is a perspective view of a first detail of the system of FIG. 14 in two different operating positions;

FIG. 19 is an exploded perspective view of the detail of FIG. 18; FIG. 20 is a perspective view of a second detail of the system of FIG. 14;

FIG. 21 is a partially exploded perspective view of the detail of FIG. 20;

FIG. 22 shows side and top views of a detail of the system of FIG. 14 in an operation sequence.

### DETAILED DESCRIPTION OF EXEMPLARY EMBODIMENTS

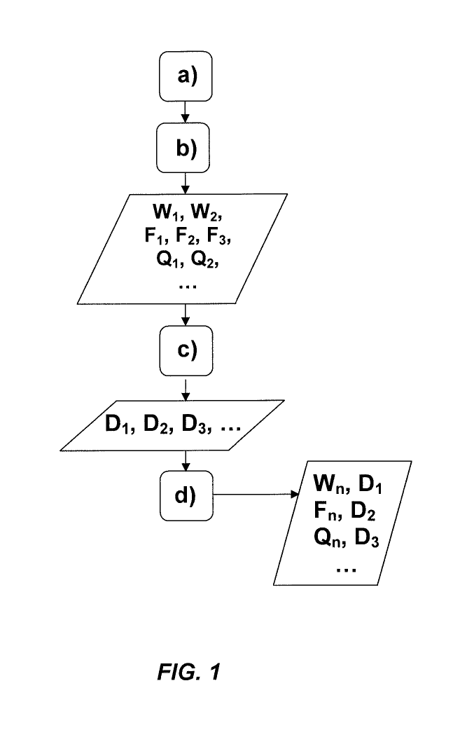

FIG. 1 schematically shows a method for optimized selection of a bicycle saddle from a population P of different saddles, provided according to special geometrical and/or functional characteristics.

As used herein, the term “provided” and its derivatives means that the saddles have been previously prepared and may be physically available in a store or other business premises for immediate delivery to the user, or be simply offered in a catalogue, for delivery upon order.

As shown in FIG. 12, each saddle in the population P generally has a substantially rigid shell T with a pad, not shown, associated therewith, as well as a protective cover C defining a seating surface.

The shell T with the cover X defines a longitudinal axis L and has a typical plan shape defining an enlarged rear portion R at which the saddle has its maximum width and a tapered front portion F, or nose, of minimum width. The front portion F is connected to the rear portion R via a central connecting portion M having curved side edges W with outwardly facing concavities and a predetermined radius of curvature r.

Furthermore, one or more saddles Sᵢ of the population P may have a hole H, at least at its central portion M, which extends through all the parts of the saddle Sᵢ, from the shell F to the cover C, or possibly a superficial hollow, whose known purpose is to reduce the pressure exerted by the saddle on the crotch of the user.

Bearing this in mind, the selection method of the invention comprises a preliminary step a) of providing a database B composed of a set Ω of physical elements, whose general element is generally referenced Sᵢ, corresponding to the different bicycle saddles of the population P.

The method includes a second step b) of subdividing the set Ω into subsets according to predetermined geometrical and/or functional characteristics. Conveniently, this subdividing step b) may be obtained by subdividing the set (Ω) into first subsets W₁, W₂ and second subsets F₁, F₂ according to the geometrical and/or functional characteristics of the saddle.

Particularly, the geometrical and/or functional features that are considered to partition the saddles Sᵢ of the database B into subsets Wₙ, Fₙ are at least the maximum width Uₘₐₓ, the presence of holes and/or superficial hollows of the saddle and, if any, the dimension a of these holes and/or hollows and their plan shape.

The method includes a third step c) of collecting a plurality of anatomical parameters of the user, particularly at least a first anatomical value I and a second anatomical value a for determining respective data D₁, D₂.

In a simple implementation of the method, the first collected anatomical value I is the intratrochanteric distance of the user, that is the distance between the greater trochanters of each femur of the user.

This measurement was found to be strictly associated with the overall size of the pelvis, whereby as the intertrochanteric distance I increases, a saddle shall be provided in which the rear portion R, having the maximum width Uₘₐₓ has a relatively high value.

Therefore, the first numerical value D₁ may be given by this measured distance I, whereby the first subset W₁, W₂ of physical elements will be represented by saddles S₁ having an optimized width Uₘₐₓ according to the first value D₁.

The second anatomical parameter a collected at step c) will be the inclination angle of the sacral plate of the user, the second value D₂ being associated with such angle.

A peculiar feature of the invention is that the anatomical parameters are collected from a user in a standing position with straight legs, which means that no effort is required from the user, and that no particular bearing or support structure is needed.

The method further comprises a step d) of selecting one of said subsets W₁, W₂; F₁, F₂ from the set Ω provided at the start.

The selected subset comprises the saddles having a first characteristic optimized according to the corresponding values D₁, D₂ . . . .

Conveniently, the selection step d) may include the selection of one of the first subsets W₁, W₂ having a first characteristic optimized according to the first value D₁ and the selection of one of the second subsets F₁, F₂ having a second characteristic optimized according to the second value D₂.

At the end of the steps a) to d), one or more saddles Sᵢ having geometrical and functional characteristics optimized for a particular user may be singled out from the initially provided set Ω.

Alternatively, the method may provide a group of different saddles, but having the above characteristics in common, which will give user a wider choice, also based on additional requirements such as cost or aesthetics.

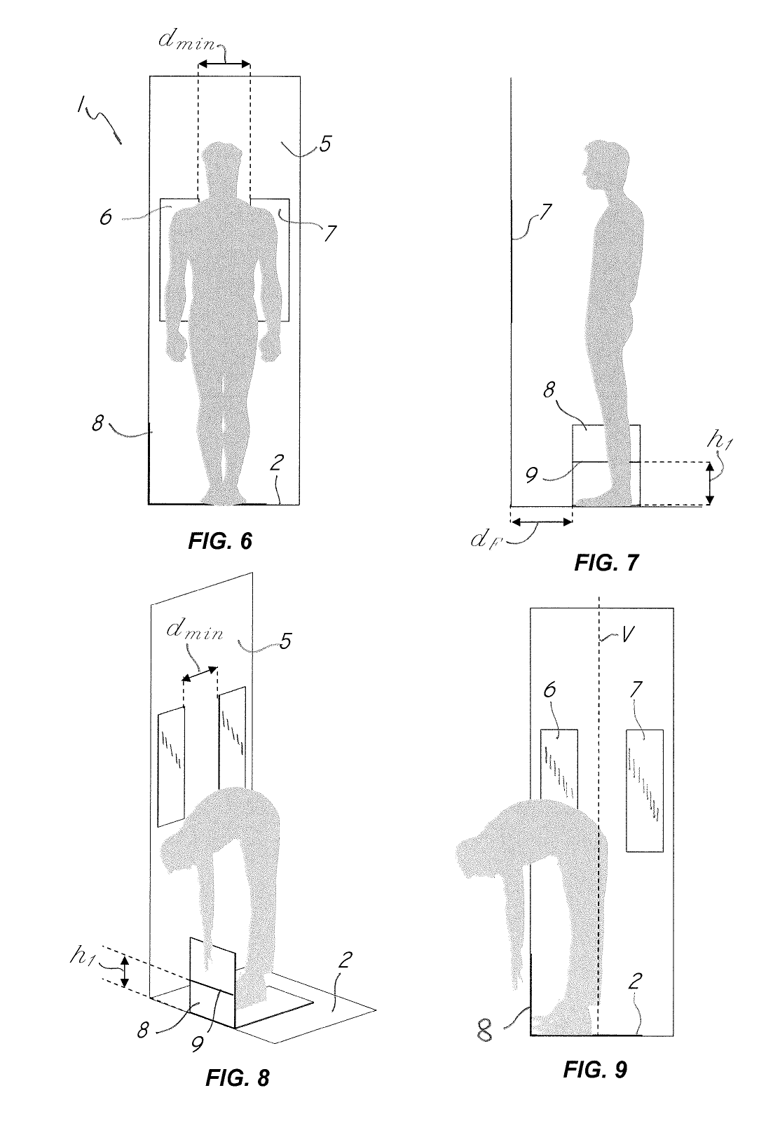

FIGS. 6 to 9 show a first system 1 for the user to autonomously carry out the step c) of the method for optimized selection of a saddle, as described above. This first system 1 is particularly suitable for selecting a saddle for non-competitive sports and/or tourist and/or urban use, e.g. in trekking bikes and/or citybikes.

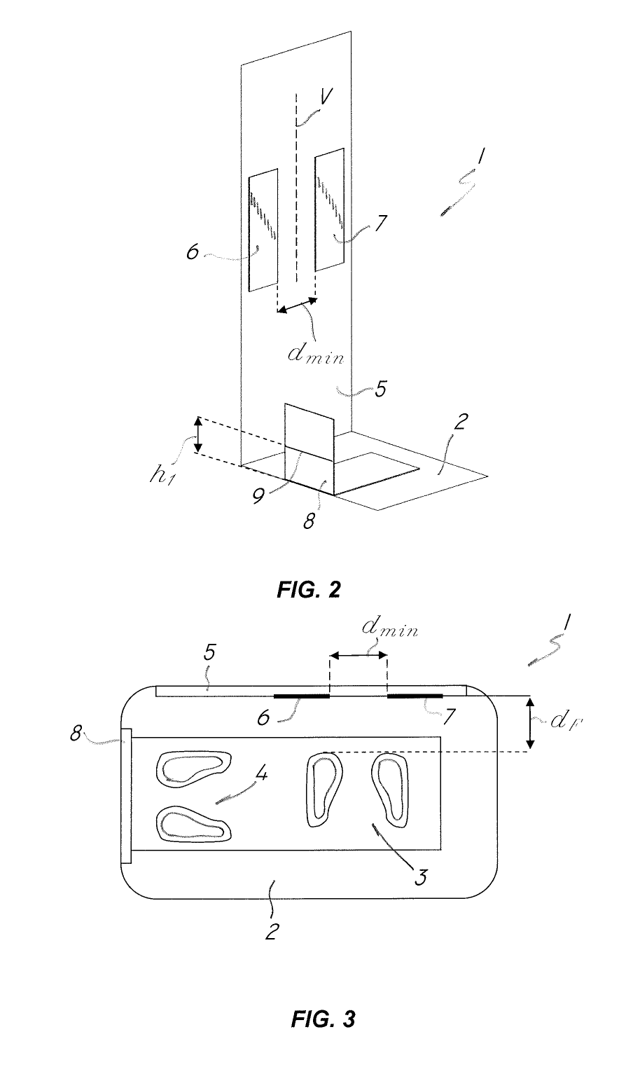

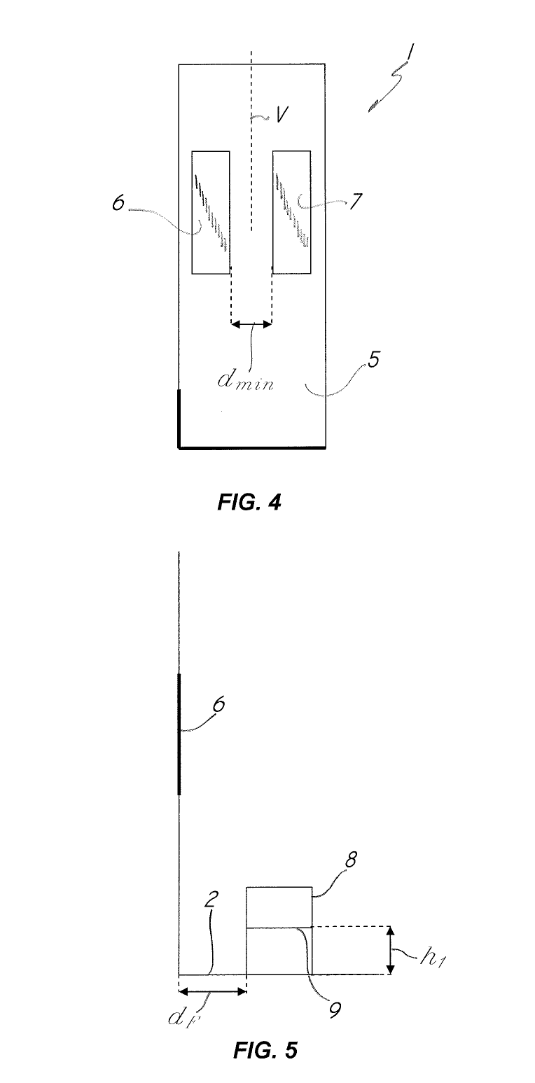

As shown in FIGS. 2 to 5, the system 1 comprises a structure consisting of a base or platform 2 in which two pairs of footprints or shapes 3, 4 are imprinted or applied to define the position of the user's feet during acquisition of the parameters I and α. A vertical plate 5 is fixed to the platform and has a pair of mirrors 6, 7 disposed at a minimum distance dₘᵢₙ from each other and with a vertical center line V.

The step c) of collecting the first anatomical parameter I may be performed by placing a standing user at a predetermined front distance d_{F} from the pair of mirrors 6, 7 to detect the profile of his/her hips with respect to the mirrors 6, 7.

No reflection of the profile on the pair of mirrors 6, 7 corresponds to a first value D₁ associated with one of the first subsets W₁, W₂, i.e. a small size of the maximum width Uₘₐₓ of the saddle.

Conversely, the reflection of the profile on the pair of mirrors 6, 7 corresponds to a first value D₁ associated with the other of the first subsets W₁, W₂, i.e. a large size of the maximum width Uₘₐₓ of the saddle.

Experimental tests showed that, when the user is placed at a front distance d_{F} of about 30 cm from the plate and the mirrors 6, 7 are at a minimum distance dₘᵢₙ of about 24 cm from each other, the limit value of the intertrochanteric distance I that can generate a reflection is about 34 cm.

Therefore, if the intertrochanteric profile is not reflected by the mirrors 6, 7, then the first value D₁ associated with one of the first subsets W₁, W₂ corresponds to small size of the saddle.

If the intertrochanteric profile is reflected by the mirrors 6, 7, then the first value D₁ associated with the other of the first subsets W₁, W₂ corresponds to a large size of the saddle.

The system 1 further comprises an additional vertical panel 8 joined to the platform 2 and placed at the side of the plate 5. The panel 8 has a horizontal landmark 9 placed at a predetermined height h₁ from the ground.

The step c) of detecting the second anatomical parameter a may be obtained by having the user bent the torso with arms stretched downwards and placed the hand tips before the panel comprising the landmark 9.

Failed attainment of the horizontal landmark 9 by the user's hand tips determines the second value D₂ associated with one of the second subsets F₁, F₂, corresponding to a saddle without holes and/or superficial hollows H.

Conversely, the attainment of the horizontal landmark 9 by the user's hand tips determines the second value D₂ associated with the other of the second subsets F₁, F₂, corresponding to a saddle having holes and/or superficial hollows H.

Conveniently, the horizontal landmark 9 may be placed at a height from the ground h₁ of 20 cm or so.

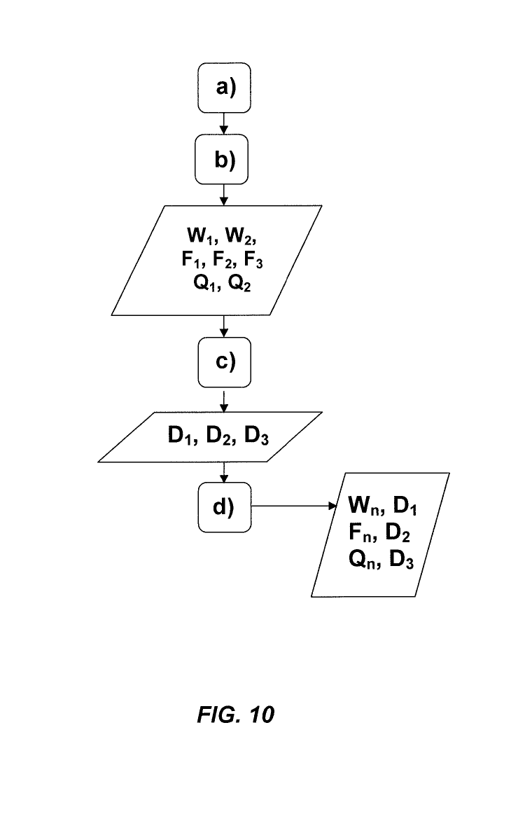

FIG. 10 schematically shows a second method for optimized selection of a saddle from a population P of competition saddles.

In the step b) of the second method the set Ω is partitioned into first subsets W₁, W₂, second subsets F₁, F₂, F₃ and third subsets Q₁, Q₂ according to geometrical and/or functional characteristics.

The step c) may include collection of a first anatomical parameter I of the user selected from the group comprising at least the intertrochanteric distance to obtain the first value D₁, a second anatomical parameter a of the user selected from the group comprising the inclination angle α of the sacral plate to obtain the second value D₂ and of a third anatomical parameter Lₘ of the user selected from the group comprising the average length of the peripheral extent of the thighs to obtain a third value D₃.

Conveniently, the selection step d) includes the selection of one of the first subsets W₁, W₂ having a first characteristic optimized according to the first value D₁, the selection of one of the second subsets F₁, F₂, F₃ having a second characteristic optimized according to the second value D₂, and the selection of one of the third subsets Q₁, Q₂ having a characteristic optimized according to the third value D₃.

Furthermore, the geometrical and/or functional characteristics that are considered to partition the saddles Sᵢ of the database B into subsets Wₙ, Fₙ, Qₙ are the maximum width Uₘₐₓ, the presence of holes and/or superficial hollows H of the saddle S and, if any, their dimension a and their plan shape.

The third anatomical parameter Lₘ may consist of the length of the average extent of the user's thighs as detected from the gluteal fold and the third subset Qₙ will comprise the saddles S₃ of the second selected subset Fₙ, whose plan shape is optimized according to the third value D₃.

Once again, all the anatomical parameters I, α, Lₘ will be collected from the user in a standing position, or with his/her torso bending forward, depending on the parameter to be measured, but never in a sitting position.

The saddles of the third subset Qₙ from which selection may be made, may differ regarding one or more of the above mentioned geometrical and/or functional characteristics, but will not be excessively differentiated from one another, thereby allowing selection to be made from a wider range, while still allowing anthropometric selection of a saddle having optimized characteristics.

Pressure of the crotch on the saddle was experimentally found to be affected by different rotations of the pelvis in the sagittal plane and not by different inclinations of the torso, such pressure causing discomfort and even pain.

Therefore, the second selection method, as shown in FIG. 10, will measure pelvic anteversion from the Adams test position. This position allows the sacral plate to be exposed, allowing determination of its inclination, which strongly depends on the extension of ischiocrural muscles, i.e. the rear muscles of the thigh belonging to the rear muscle chain.

In a preferred embodiment of the method, the measured average length of the peripheral extent of the thigh will be related to the measured value of the intertrochanteric distance I that was previously detected for the same user using a predetermined formula or algorithm, to obtain an anatomical index XQ, which will allow optimal selection of the third subset Qₙ of saddles S₃ which, as mentioned above, may also consist of a single saddle.

Particularly, the anatomical index XQ is proportional to the ratio of the intertrochanteric distance I and the average length of the average extent of the thighs of the user, which is measured from the gluteal fold, less a corrective constant designed to account for the male and female anatomy.

The value of this index XQ may be used to select saddles having standard plan shapes or saddles having a narrow connecting portion M with concave side edges E and a sufficiently small maximum transverse width G, slightly greater than the maximum width of the means for connection of the saddle to the seat post, not shown, such that a narrow portion is defined at the user's thighs, for reducing compression and rubbing of the inner surface of the thighs against the lateral edges E and for allowing the rider's position to move toward smaller-width saddle portions, to facilitate extension of the thigh on the pelvis.

This will ensure contact of ischial tuberosities with the saddle pad, which is the maximum comfort area, where the saddle is larger and padded.

By way of example and without limitation, saddles may be as disclosed in the International patent WO2012010988. Saddles of this type are particularly suitable for users having a relatively great average length of the thigh extent with respect to their build, and hence a lower anatomical index XQ, for whom a standard-profile saddle would cause excessive rubbing of the interior of the thigh and restrict the extension of the thigh from the pelvis.

Conversely, narrow-profile saddles will increase both comfort and pedaling performance even for users with a small anatomical index XQ who might need a saddle with a relatively small maximum width Uₘₐₓ.

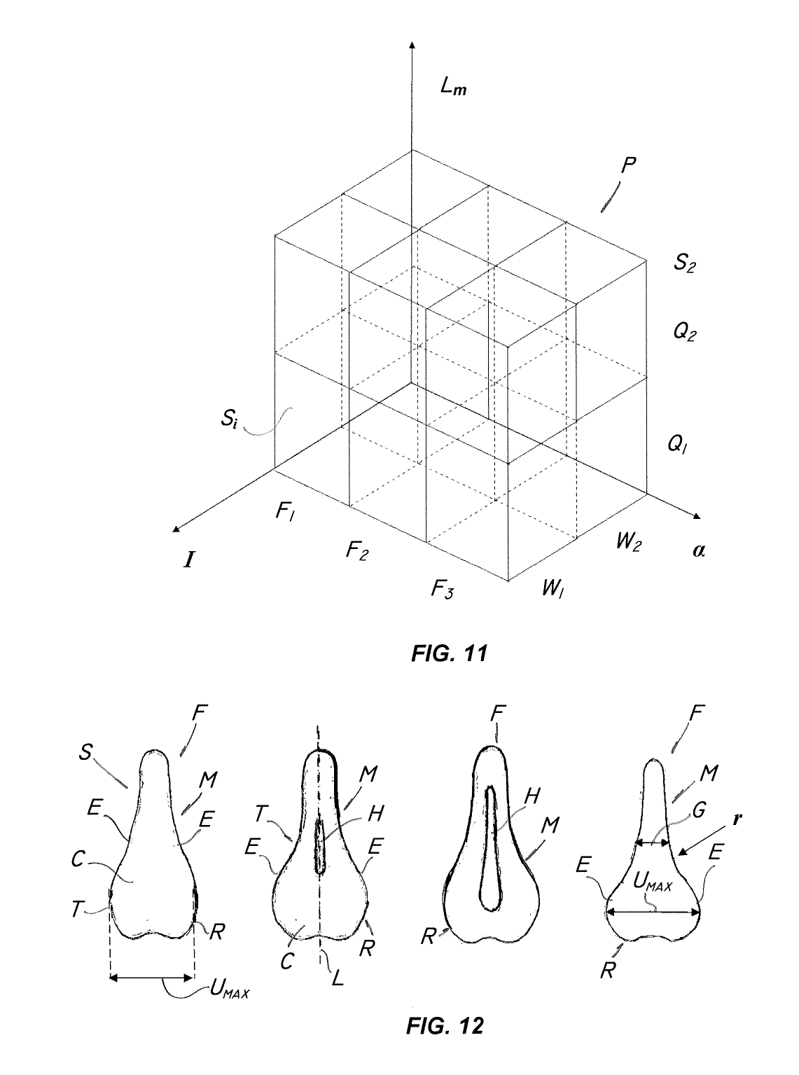

FIG. 11 schematically shows a matrix or diagram of three-dimensional distribution of the database B of physical elements of the saddles that have been provided, as belonging to the population P.

The set Ω may define a matrix that can be represented as a three-dimensional diagram having said anatomical parameters I, α, Lₘ, as coordinate axes, as schematically shown in FIG. 11.

For example, the set may be partitioned into two first subsets W₁, W₂ according to the maximum width value Uₘₐₓ that can be detected at the wider rear portion W. One of these first subsets, referenced W₁ comprises saddles Sᵢ having a smaller maximum width Uₘₐₓ than that of the other first subset W₂, which will include saddles Sᵢ having maximum widths Uₘₐₓ in a range of relatively high values.

Preferably, there may be a first subset W₁ having saddles Sᵢ with a maximum width of a predetermined smaller value, for example substantially close to 130 mm, whereas the other first subset W₂ will comprise saddles having a maximum width Uₘₐₓ of a predetermined greater value, for example substantially close to 140 mm.

The saddles Sᵢ of the first subset with the smaller maximum width value W₁ are deemed to be optimized for users having an intertrochanteric distance I smaller than a predetermined reference value, i.e. substantially ranging from 310 mm to 350 mm, preferably from 330 mm to 340 mm, and more preferably of about 336 mm.

The saddles Sᵢ of the first subset W₂ with the greater maximum width value Uₘₐₓ are deemed to be optimized for users having an intertrochanteric distance I greater than said reference value.

It shall be understood that more than the two first subsets with different maximum widths Uₘₐₓ of the saddles may be defined in the set 0, to improve the optimization degree.

Each of the first subsets W₁, W₂ may have two or more second subsets F₁, F₂, defined therein, differing in the presence and/or dimension a of a hole H or a superficial hollow of the saddle.

For example, each of the first subsets W₁, W₂ may comprise three second subsets F₁, F₂, F₃, one F₁ comprising saddles Sᵢ without holes and/or hollows H, another F₂ comprising saddles Sᵢ with a hole H having a relatively small plan dimension a, and the last F₃ comprising saddles Sᵢ with a hole H having a relatively large plan dimension.

The saddles Sᵢ with no hole H are particularly suitable for users for whom the detected angle of rotation α is smaller than a predetermined minimum value, e.g. ranging from 40° to 55°, preferably from 45° to 52° and more preferably substantially of about 49°.

The saddles Sᵢ with a hole or superficial hollow H having a relatively small dimension a are particularly suitable for users for whom the detected angle of rotation α ranges from the above mentioned predetermined minimum value and a predetermined maximum value, e.g. from 55° to 75°, preferably from 60° to 70° and more preferably substantially of about 67°.

The saddles Sᵢ with a hole or superficial hollow H having a relatively large dimension a are particularly suitable for users for whom the detected rotation angle α is greater than said predetermined maximum value.

Thus, a user, either a man or a woman, having a high inclination angle α of the sacral plate, involving the risk of excessive compression exerted on the crotch, will tend to select a saddle Sᵢ having a relatively large hole H, ensuring the presence of an appropriately sized area where no compression is exerted on the crotch.

Finally, each of the second subsets F₁, F₂, F₃ may have two or more third subsets Q₁, Q₂, . . . defined therein, differing in the plan shape of the saddles.

For example, two third subsets Q₁, Q₂, may be defined, one of which Q₁ comprises standard-profile saddles Sᵢ, whereas the other third subset Q₂ will comprise narrow-profile saddles Sᵢ.

Standard-profile saddles Sᵢ are deemed to be optimal for a user having an anatomical index XQ that is higher than a predetermined reference value XQᵣ, e.g. ranging from 3 to 4, preferably from 3.5 to 3.8 and more preferably of about 3.72.

Narrow-profile saddles Sᵢ are deemed to be optimal for a user having an anatomical index XQ that is lower than said reference value.

In each subset, saddles may be further differentiated according to further geometrical and/or functional characteristics.

For example, each saddle Sᵢ may be associated with a parameter Cₜ adapted to assess its comfort degree, which may result from the combination of multiple physical, i.e. either chemical or mechanical properties, in either static or dynamic conditions.

For example, a parameter Cₜ may be established as a function of bending strength at three points, expressed as N, resulting from a 5 mm displacement imparted at the point BRP®, substantially coinciding with the axial position of the saddle in which its width is about 70 mm, of the thickness of the finished saddle pad coinciding with the ischial region, i.e. the rear portion designed for buttock support, and of the finished saddle density or hardness of the pad, as measured with a Shore LX-C durometer, at the ischial tuberosity support points.

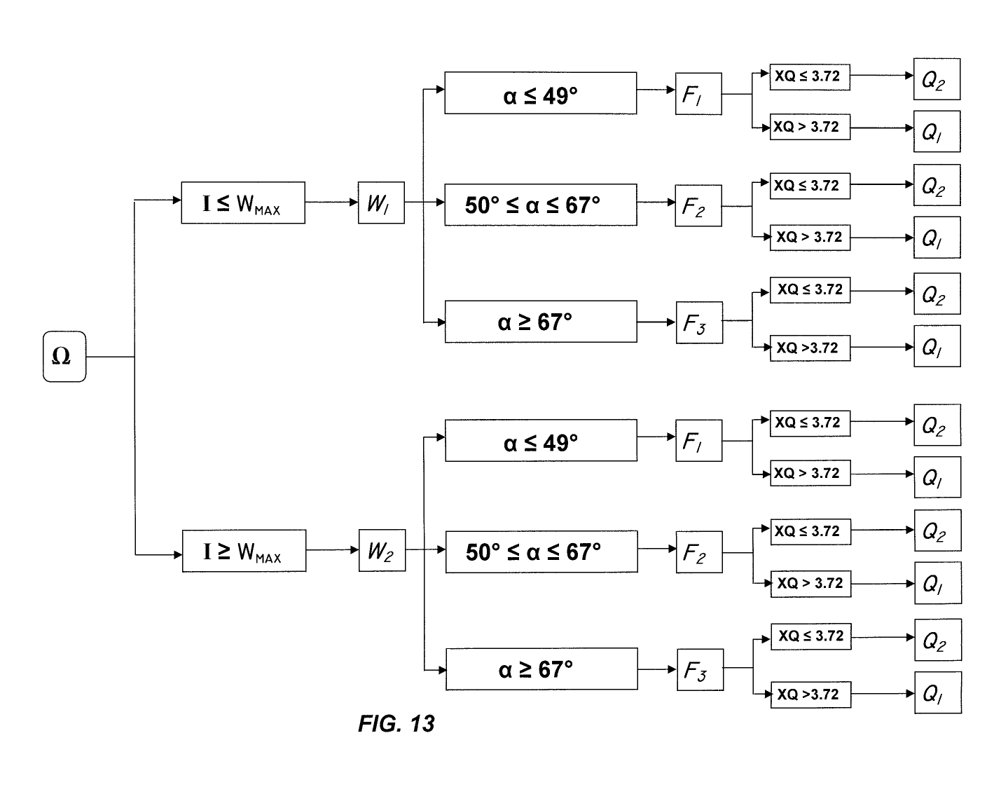

FIG. 13 shows a flow chart of a method of the invention, with the saddles arranged in subsets, according to the three-dimensional diagram of FIG. 11.

Nevertheless, it will be appreciated that the set Ω may be also organized otherwise than as described above, and two or more first subsets F₁, F₂, F₃ may be provided, differing in the hole H or two or more first subsets Q₁, Q₂ may be provided, mutually differing in their plan shape. Thus, the second and third subsets will differ in another of the remaining characteristics, according to a different combination.

As a result, the first anatomical parameter to be measured may be the inclination angle α of the sacral plate, i.e. the average length Lₘ of the peripheral extent of the thigh, instead of the intertrochanteric distance.

The second and third parameters may consist of any other of the remaining anatomical parameters, according to any possible detection sequence.

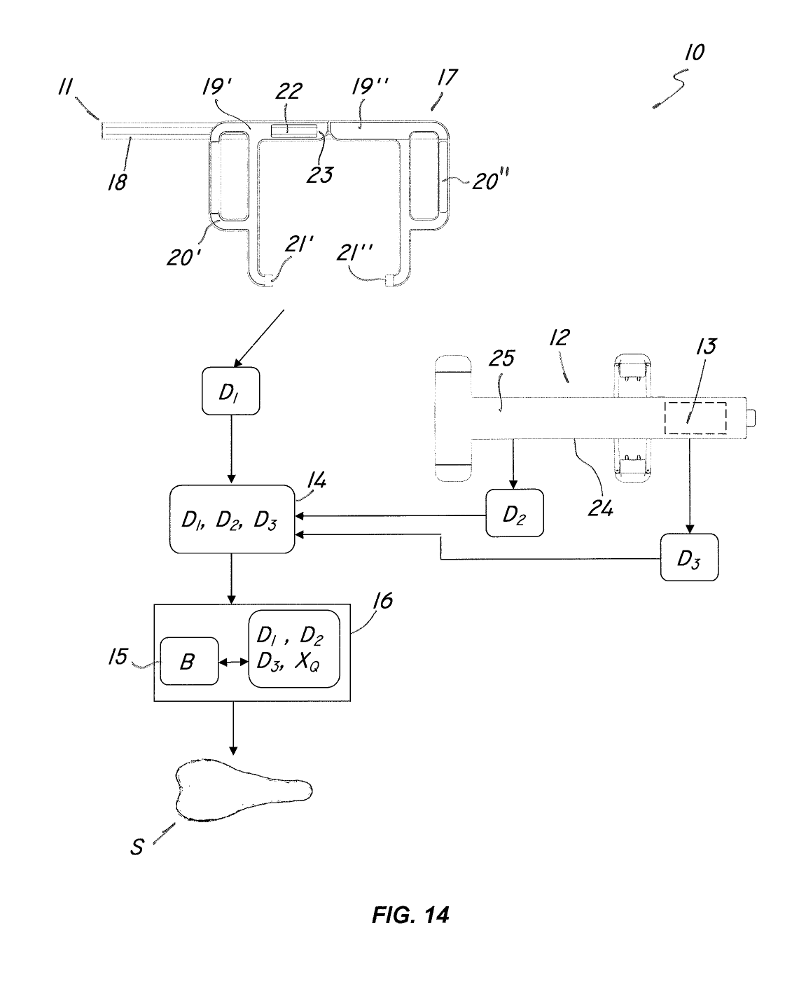

FIG. 14 shows a second system 10 for implementing the step c) of the method as shown in FIG. 10.

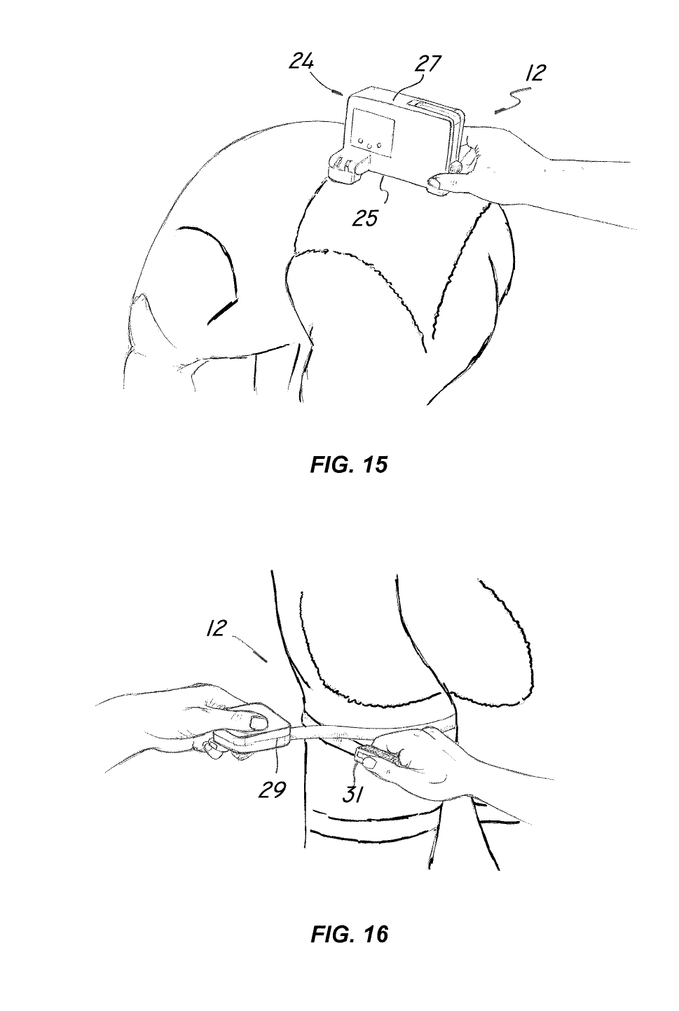

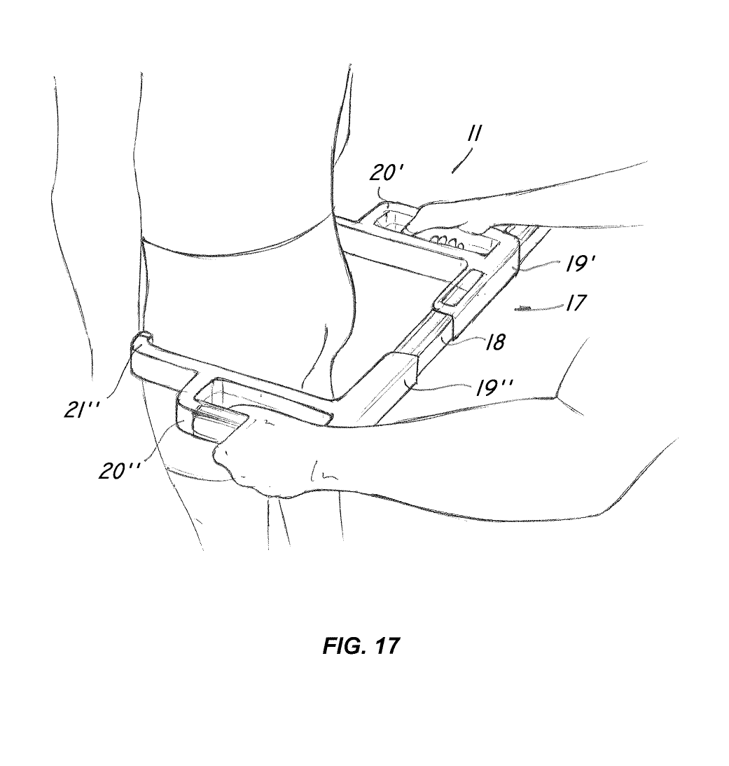

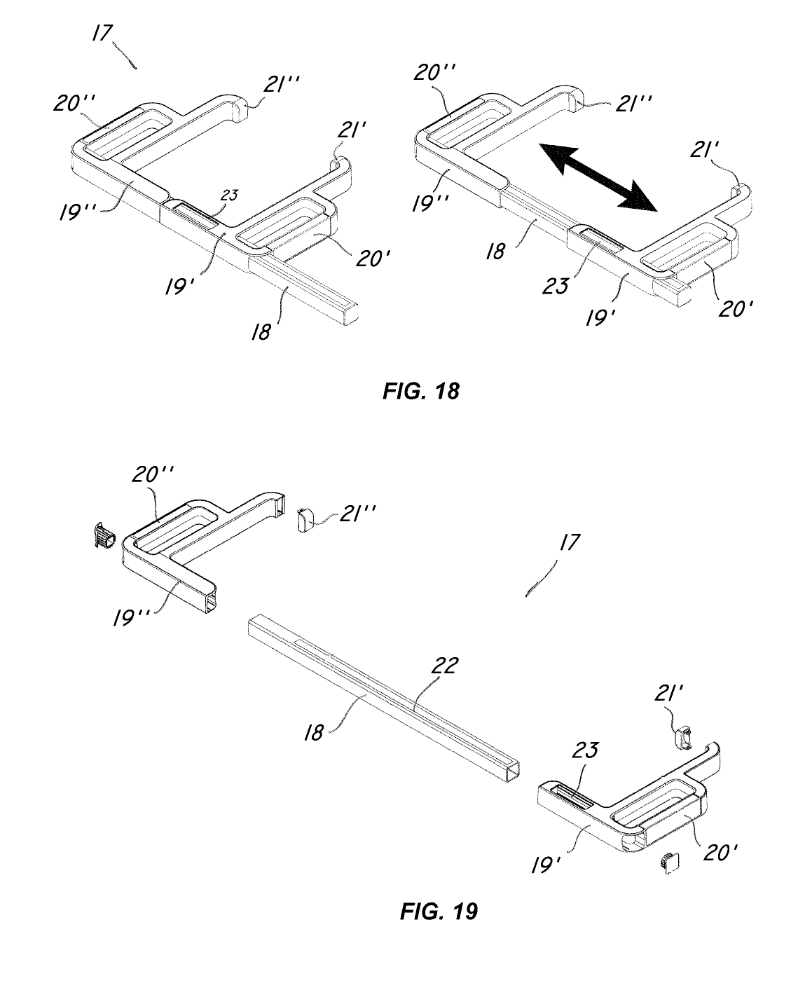

Particularly, FIGS. 15 to 17 show certain exemplary and non-limiting operating modes of the present invention, for detection of the anatomical parameters I, α, Lₘ respectively.

The second system 10 essentially comprises first measuring means 11 for detecting the first value D₁ indicative of the intertrochanteric distance I on a standing user, second measuring means 12 for detecting the second value D₂ indicative of the inclination angle α of the sacral plate in the sagittal plane of the user standing with his/her torso leaning forward, and third measuring means 12 for detecting a third value D₃ indicative of the length Lₘ of the average extent of the user's thighs, as measured in the standing position.

Converter means 14 are also provided, such as transducers for conversion of said first, second and third values D₁, D₂, D₃ into a digital format.

The system is complemented by processing means, such as conventional desktop or laptop PCs, i.e. a dedicated computer containing a microprocessor, one or more RAM and/or mass memory units and I/O interface means and an operating system, such as Windows® or Linux®.

Particularly, the memory units 15 may store both the database B of a set Ω of physical elements Sᵢ corresponding to bicycle saddles having different geometrical and/or functional characteristics, and the values D₁, D₂, D₃.

A program may be installed in the microprocessor 16, to process such digitized values D₁, D₂, D₃, thereby also allowing calculation of the above described anatomical index XQ, and automatic anthropometric selection of the user-optimized saddle from the database B.

As better shown in FIG. 14, the first measuring means 11 may include a gauge device 17 having a graduated rod 18 with end tailpieces 19′, 19″ being slidingly mounted thereto, for abutment against the hips of the standing user, at the trochanteric masses.

Preferably, the end tailpieces 19′, 19″ extend perpendicular to the rod 18 and have handgrips 20′, 20″ for an operator.

In practice, the gauge device 17 is substantially C shaped for partially encircling the user and detecting the intertrochanteric distance I.

The two tailpieces 19′, 19″ have a curved free end 21′, 21″ which is designed to abut the trochanteric masses and allow the distance I to be directly read on the graduated scale 22 associated with the rod 18 at a specially provided index. For this purpose, one of the two sliding tailpieces 19′ may be equipped with a reading window 23 having such index.

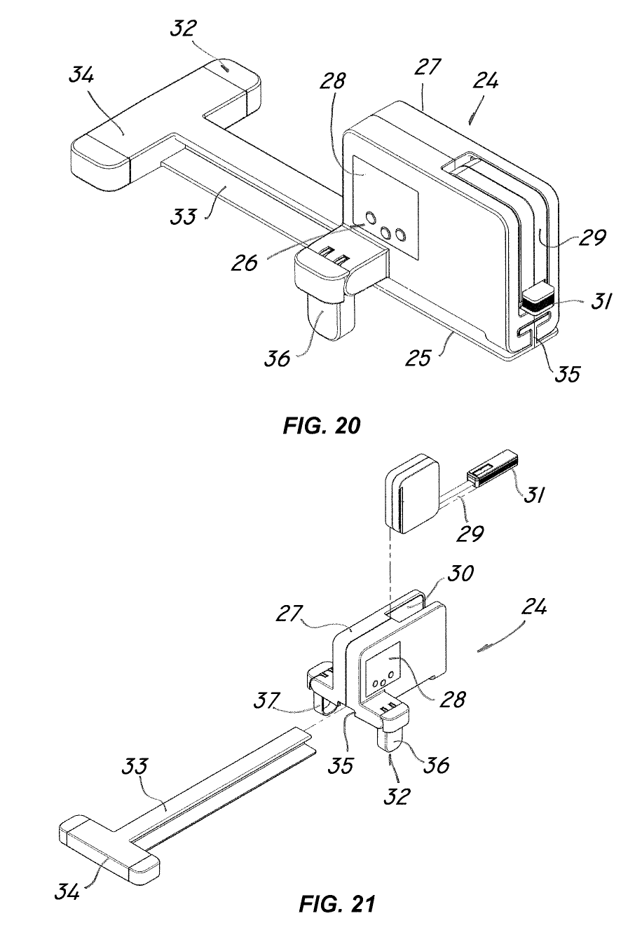

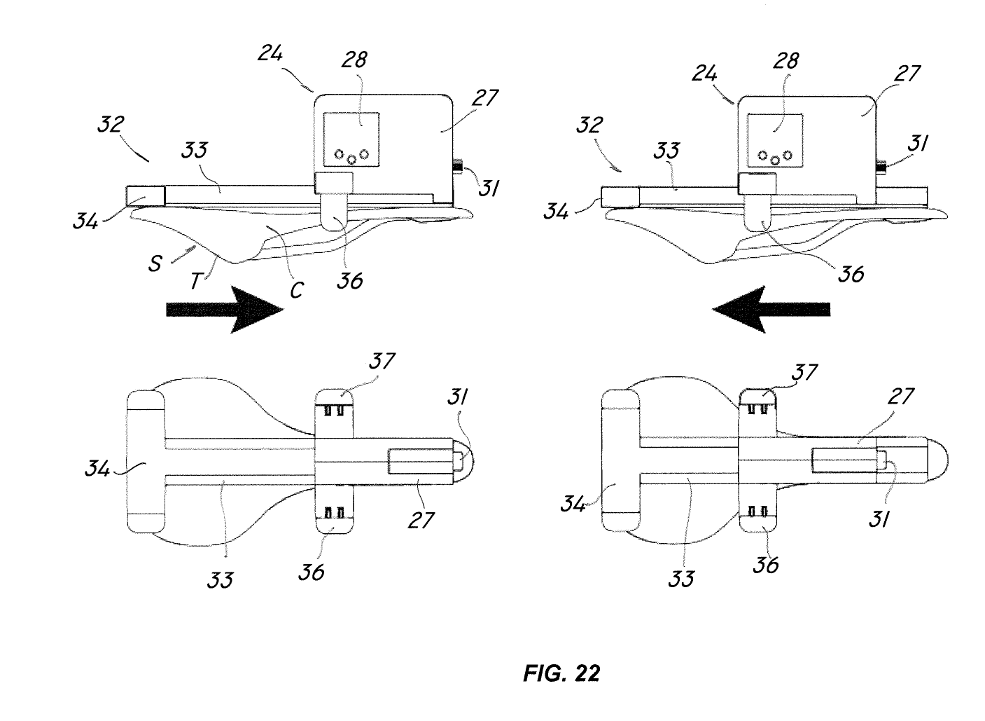

FIGS. 20 to 21 are more detailed views of an inclinometer device 24 that is part of the second measuring means 12 and is adapted to detect the inclination angle α of the sacral plate of the user.

Particularly, the inclinometer 24 comprises a base 25 which is designed to lay on the sacral plate of the user and a sensitive element for measuring the inclination angle α, not shown.

Conveniently, the base 25 is associated to indicator means 26 that show the detected measurement which, in a preferred, non-limiting configuration of the present invention, may be of digital type.

For example, the inclinometer 24 comprises a box-like casing 27 mounted above the base 25 and having an electronic card, not shown, therein, connected to a digital display 28 for displaying the detected inclination angle α.

The third measuring means 13 may include a flexible measuring ribbon 29 or any other similar measuring instrument, having a graduated scale, not necessarily with the International System scale, which is adapted to encircle the user's thigh and measure the length of its peripheral extent in the desired area.

Advantageously, the measuring ribbon 29 may be housed in a compartment 30 of the box-like casing 27 of the inclinometer 24, such that it may be unwound by overcoming the force of elastic return means, and may have a calibrated dynamometer 31 for measuring with at a predetermined constant and repeatable tension.

In a particularly advantageous aspect, the invention provides an accessory 32 adapted to be associated with the inclinometer 24 and to measure the inclination of a saddle S for adjustment thereof.

Particularly, the accessory 32 comprises a rod 33 with an end crosspiece 34, adapted to be slidingly received in a lower central groove 35 formed in the base 25 of the inclinometer 24.

Thus, the rod 33 will have three distinct support points, allowing the accessory 32 to be used on any kind of saddle, even on saddles having a seating surface with particularly deep concavities and hollows.

The rod 33 comprises a pair of retractable tailpieces 36, 37 symmetrically arranged with respect to the rod 33.

The rod 33 is designed to be laid on the top surface C of a saddle S, with the tailpieces 36, 37 designed to abut the side edges E of the saddle proximate to its narrow section M.

Preferably, in the operating extracted position of the two tailpieces, the latter have a predetermined transverse distance therebetween of about 70 mm, thereby defining a 70 mm-width area in the saddle, which corresponds to the BRP point (Biomechianical Reference Point) required for the above mentioned comfort index determining test.

Furthermore, the tailpieces 36, 37 are configured to prevent insertion of the rod 33 when the tailpieces 36, 37 are in the retracted position, thereby preventing the use of the inclinometer 24 for measuring the inclination of the saddle S when the inclinometer 24 itself is in a configuration adapted for measurement of the inclination angle α of the sacral plate.

Advantageously, the measuring ribbon 29 that is housed in the compartment 30 of the box-like enclosure 27 may be used, if required, to measure the distance of the saddle S from the handlebar, at the same time as the inclination of the saddle S is being measured

Therefore, the accessory 32 associated with the inclinometer 24 and with the measuring ribbon 29 is a multifunctional instrument that allows both anthropometric selection of the best saddle for a user and achievement of an optimal adjustment of the angular position of the saddle and its longitudinal position with respect to the frame of the bicycle.

Finally, the gauge device 17 may be used for to measure the width of the user's shoulders, to also afford optimal selection of a handlebar from a number of handlebars having different widths.

Therefore, the system so configured is a multifunctional system that allows customization of the saddle and most of the bicycle frame, thereby providing an anthropometrically optimized bicycle.

The method and system of the invention are susceptible to a number of changes or variants, within the inventive concept disclosed in the appended claims. All the details thereof may be replaced by other technically equivalent parts, and the materials may vary depending on different needs, without departure from the scope of the invention.

For example, all the above described measuring means 11, 12, 13 may be replaced by other means allowing the required measurements to be made, and may be of either analog or digital type, possibly of electronic or laser measurement type, or the like.

While the method and system have been described with particular reference to the accompanying figures, the numerals referred to in the disclosure and claims are only used for the sake of a better intelligibility of the invention and shall not be intended to limit the claimed scope in any manner.

1. A method for optimized selection of a bicycle saddle in a population of different saddles, comprising the following steps:
a) providing a data base (B) formed by a set (Ω) of physical elements (Sᵢ) corresponding to the population of different saddles;
b) subdividing said set (Ω) into a plurality of subsets (W₁, W₂; F₁, F₂; Q₁, Q₂; . . . ) as a function of one or both of predetermined geometrical or functional characteristics;
c) collecting a plurality of anatomical parameters (I, α, Lₘ, . . . ) of a user for determining corresponding data (D₁, D₂, D₃, . . . ); and
d) selecting one of said subsets (W₁, W₂; F₁, F₂; Q₁, Q₂; . . . ) having corresponding optimized characteristics according to said data (D₁, D₂, D₃, . . . );
wherein said geometrical or functional characteristics comprise at least a maximum width (Uₘₐₓ), presence and dimension of holes or superficial hollows (H), and plane shape of the saddles; and
wherein said plurality of anatomical parameters (I, α, Lₘ, . . . ) is collected from the user in a standing position and with stretched legs.

2. The method as claimed in claim 1, wherein the step of subdividing is carried out by subdividing said set (Ω) in first subsets (W₁, W₂) and second subsets (F₁, F₂) according to said geometrical or functional characteristics.

3. The method as claimed in claim 2, wherein the step of collecting comprises collecting a first anatomical parameter (I) of the user chosen from a group including at least an intertrochanterical distance (I) for obtaining a first data (D₁), and a second anatomical parameter of the user chosen from a group including an inclination angle (α) of a sacral plate for obtaining a second data (D₂).

4. The method as claimed in claim 3, wherein the step of selecting comprises selecting one of said subsets (W₁, W₂) having a first optimized characteristic on a strength of said first data (D₁) and selecting one of said second subsets (F₁, F₂) having a second optimized characteristic according to said second data (D₂).

5. The method as claimed in claim 3, wherein said first anatomical parameter (I) is the intertrochanterical distance of the user and said first subsets (W₁, W₂) comprise elements (Sᵢ) having the maximum width (Uₘₐₓ) that is optimal according to said first data (D₁).

6. The method as claimed in claim 3, wherein said inclination angle (α) defining said second anatomical parameter is detected by the user with upstanding legs and with torso bent forward, and wherein said second subsets (F₁, F₂) comprise optimized elements (Sᵢ) having or not holes or superficial hollows (H) according to said second data (D₂).

7. The method as claimed in claim 3, wherein the step of collecting said first anatomical parameter (I) is performed with the user in standing position at a predetermined front distance (d_{F}) with reference to a pair of mirrors (6, 7) placed on a vertical plate (5) at a reciprocal minimum distance (dₘᵢₙ) and with a vertical centerline (V), and further by detecting user profile with respect to said pair of mirrors (6, 7).

8. The method as claimed in claim 7, wherein no reflection of the user profile on said mirrors (6, 7) corresponds to said first data (D₁) associated to said first subset (W₁, W₂), corresponding to a smaller size of the maximum width (Uₘₐₓ) of the saddle, while the reflection of the profile on said mirrors (6, 7) corresponds to said first data (D₁) associated to said first subset (W₁, W₂), corresponding to a larger size of the maximum width (Uₘₐₓ) of the saddle.

9. The method as claimed in claim 3, wherein the step of collecting said second anatomical parameter (α) is performed with the user bending the torso with arms stretched downward and placing hands tips in front of a vertical panel (8) having an horizontal landmark (9) placed at a predetermined height (h₁) from the ground.

10. The method as claimed in claim 9, wherein a failed attainment of said horizontal landmark (9) by ends of hands of the user corresponds to a second data (D₂) associated to said second subset (F₁, F₂) and corresponding to a saddle without holes or superficial hollows (H), whereas attaining said horizontal landmark (9) by the hand tips of the user corresponds to the second data (D₂) associated to said second subset (F₁, F₂) and corresponding to a saddle having holes or superficial hollows (H).

11. The method as claimed in claim 1, wherein the step of subdividing is performed by subdividing said set (Ω) in first subsets (W₁, W₂), second subsets (F₁, F₂, F₃) and third subsets (Q₁, Q₂) according to said geometrical or functional characteristics.

12. The method as claimed in claim 11, wherein the step of collecting comprises detecting a first anatomical parameter (I) of the user chosen from a group including at least an intertrochanterical distance (I) for obtaining a first data (D₁), a second anatomical parameter (α) of the user chosen from a group including an inclination angle (α) of the sacral plate for obtaining a second data (D₂), and a third anatomical parameter (Lₘ) of the user chosen from the group including an average length of a peripheral extent of user thighs for obtaining a third data (D₃).

13. The method as claimed in claim 12, wherein the step of selecting comprises selecting one of said first subsets (W₁, W₂) having a first optimized characteristic according to said first data (D₁), selecting one of said second subsets (F₁, F₂, F₃) having a second optimized characteristic according to said second data (D₂), and selecting one of said third subsets (Q₁, Q₂) having a third optimized characteristic according to said third data (D₃).

14. The method as claimed in claim 13, wherein the average length (Lₘ) of the peripheral extent of the user thighs defining a third anatomical parameter is detected at a gluteal fold of the user placed in a standing position, and wherein said third subsets (Q₁, Q₂) comprise elements (Sᵢ) having an optimized plan shape according to said third data (D₃).

15. The method as claimed in claim 14, wherein said third subsets (Q₁, Q₂) are determined through an anatomical formula or algorithm (XQ) obtained as a function of a ratio between the intertrochanterical distance (I) and the average length of the peripheral extent of the user thighs collected at the gluteal fold.

16. The method as claimed in claim 15, wherein said set (Ω) of physical elements (Sᵢ) defines a matrix arranged to be represented with a tridimensional diagram having as coordinated axes said first (I), said second (α), and said third (Lₘ) anatomical parameters.

17. A system for anthropometric selection of an optimized bicycle saddle, comprising:
a first measurement device (11) configured to detect a first anatomical parameter (I) of a user;
a second measurement device (12) configured to detect a second anatomical parameter (α) of the user;
a third measurement device (13) configured to detect a third anatomical parameter (Lₘ) of the user;
a converter (14) converting said first, second, and third anatomical parameters (I, α, Lₘ) into first, second and third numerical data (D₁, D₂, D₃);
a memory (15) storing a database (B) formed by a set (Ω) of physical elements (Sᵢ) corresponding to saddles with one or both of different geometrical or functional characteristics and memorizing said first, second and third numerical data (D₁, D₂, D₃); and
a processing and computing unit (16) configured to calculate said first, second, and third numerical data (D₁, D₂, D₃) and to select one or more optimized saddles in said database (B) according to claim 11.

18. The system as claimed in claim 17, wherein said first measurement device (11) comprises a gauge device (17) with a graduated rod (18) having end tailpieces (19′, 19″) that are longitudinally slidable and are designed to abut against hips of the user in standing position proximate to intertrochanterical masses, wherein said end tailpieces (19′, 19″) extent perpendicularly to said rod (18) and have handgrips (20′, 20″) for an operator.

19. The system as claimed in claim 18, wherein said second measurement device (12) comprises an inclinometer (24) with a base (25) designed to lay on a sacral plate of the user with the user's torso bent forward.

20. The system as claimed in claim 19, wherein said inclinometer (24) is a digital-device and comprises a box-shaped casing (27) disposed above said base (25) and enclosing an electronic card connected to a digital display (28) for displaying an inclination angle (α).

21. The system as claimed in claim 20, wherein said third measurement device (13) comprises a measuring ribbon (29) made from a flexible material, housed in a compartment (30) of said box-shaped casing (27), said measuring ribbon (29) having a graduated scale for providing a measurement of a length of a peripheral extent of each thigh, and a free end with a calibrated dynamometer (31) for repeating said measurement with a predetermined tension.

22. The system as claimed in claim 21, wherein an accessory (32), configured to be coupled with said inclinometer (24) for adjusting the inclination of the bicycle saddle, wherein said accessory (32) comprises a rod (33) with an end cross piece (34) configured to be slidably housed in a central groove (35) of said base (25) and a pair of retractable tailpieces (36, 37) arranged symmetrically with respect to said rod (33) at a predetermined distance corresponding to a width of the saddle in correspondence of a Biomechanical Reference Point.
